(19) United States
(12) Patent Application Publication
(10) Pub. No.: US 2022/0387102 A1
(43) Pub. Date: Dec. 8, 2022

(54) MEDICAL SYSTEMS FOR ABLATING TISSUE

(71) Applicant: Boston Scientific Scimed, Inc., Maple Grove, MN (US)

(72) Inventors: Travis HENCHIE, Worcester, MA (US); Serena SCOTT, Worcester, MA (US); Kevin L. BAGLEY, Natick, MA (US)

(73) Assignee: Boston Scientific Scimed, Inc., Maple Grove, MN (US)

(21) Appl. No.: 17/774,275

(22) PCT Filed: Nov. 4, 2020
(86) PCT No.: PCT/US2020/058852

§ 371 Date: May 4, 2022

Related U.S. Application Data
(60) Provisional application No. 62/930,721, filed on Nov. 5, 2019.

(51) Int. Cl. A61B 18/14; A61B 18/12; A61B 34/00

(52) U.S. Cl. CPC A61B 18/1492; A61B 18/1206; A61B 34/25; A61B 2018/00994

(57) ABSTRACT

A medical system may comprise a catheter (101) for ablating tissue including a flexible longitudinal body including a distal end; and a distal portion extending distally from the distal end of longitudinal body. The distal portion may include a plurality of electrodes (103). The medical system may also comprise one or more control units (112) coupled to the catheter and configured to (1) control a supply of electrical energy to each of the plurality of electrodes and (2) automatically control a position of the distal portion of the catheter.

### CROSS-REFERENCE TO RELATED APPLICATIONS

This application claims the benefit of priority from U.S. Provisional Application No. 62/930,721, filed Nov. 5, 2019, which is incorporated by reference herein in its entirety.

### TECHNICAL FIELD

Various aspects of the present disclosure relate generally to tissue ablation, including radiofrequency ablation of tissue. More specifically, at least certain embodiments of the present disclosure relate to systems, devices, and related methods for ablating tissue, among other aspects.

### BACKGROUND

Technological developments have given users of medical systems, devices, and methods, the ability to conduct increasingly complex procedures on subjects. The ablation of tissue, for example, often involves the use of devices transmitting radiofrequency energy in order to ablate the tissue. In some examples, a user may implement a radiofrequency ablation treatment algorithm governed by setting a constant power and ablation time period to treat the desired tissue. The tissue ablation zone from this method may be a rough estimate of tissue requiring treatment, as the physician may not have direct visualization during the treatment, and may have limited feedback during treatment and post treatment for confirming accurate treatment of targeted tissue. In some examples, such a treatment algorithm may result in an increase in the number of injuries related to electrosurgery. For example, a portion of healthy tissue may inadvertently be ablated. There is a need for electrosurgical devices and systems that address this and/or other difficulties.

### SUMMARY

Aspects of the disclosure relate to, among other things, systems, devices, and methods for ablating tissue. Each of the aspects disclosed herein may include one or more of the features described in connection with any of the other disclosed aspects.

A medical system may comprise a catheter for ablating tissue including a flexible longitudinal body including a distal end; and a distal portion extending distally from the distal end of longitudinal body. The distal portion may include a plurality of electrodes. The medical system may also comprise one or more control units coupled to the catheter and configured to (1) control a supply electrical energy to each of the plurality of electrodes and (2) automatically control a position of the distal portion of the catheter.

Any of the systems and devices disclosed herein may have any of the following features. A drive system may be configured to move a catheter proximally and distally, and the drive system may be in communication with and controlled by the one or more control units. A power generator may be coupled to and controlled by one or more control units for providing electrical energy to each of the plurality of electrodes; and a scanner may be configured to create images of a patient's anatomy. The one or more control units may be configured to monitor an impedance of each of the plurality of electrodes and adjust the electrical energy supplied to each of the plurality of electrodes based on the monitored impedance. A graphical user interface may be configured to allow a user to select an area of tissue targeted for ablation by the plurality of electrodes. The one or more control units may be configured to adjust an amount of electrical energy supplied to at least one of the plurality of electrodes based on at least one image created by the scanner. The one or more control units may include a plurality of stored ablation patterns, and each stored ablation pattern may include output energy levels for each of the plurality of electrodes. The catheter may include an internal element extending from a proximal portion of the catheter to the distal portion. The internal element may include a distal protrusion with a radially-outermost surface in contact with a radially-inner surface of the distal portion, the internal element may be positioned within, and moveable relative to, the distal portion and the longitudinal body, and the internal element may be configured to transfer electrical energy to each of the plurality of electrodes independently of others of the plurality of electrodes. The catheter may include an ultrasound probe positioned within the distal portion. The scanner may be configured to detect the position of the ultrasound probe. The distal portion of the catheter may be expandable and may include an interior portion and an exterior surface, wherein each of the plurality of electrodes extends from the interior portion to the exterior surface. The distal portion of the catheter may be cylindrical and may include a conical distal portion and a conical proximal portion; and the plurality of electrodes may form a grid pattern around the radially-outermost portion of the distal portion. The distal protrusion may be configured to activate each of the plurality of electrodes independently when in contact with each electrode, and the distal protrusion may be configured to translate longitudinally and rotate relative to the distal portion. Each of the plurality of electrodes may not be connected to a proximal lead; and the distal protrusion may be curved. The drive system may include a plurality of motors to translate the catheter longitudinally and to rotate the catheter about a longitudinal axis of the catheter. The one or more control units may be configured to independently supply electrical energy to each of the plurality of electrodes.

In another example, a medical system may comprise a catheter for ablating tissue including a flexible longitudinal body including a distal end; and a distal portion extending distally from the distal end of longitudinal body, the distal portion including a plurality of electrodes. The medical system may also comprise one or more control units coupled to the catheter and configured to (1) supply electrical energy to each of the plurality of electrodes independently and (2) automatically control a position of the distal portion of the catheter. The medical system may further comprise a drive system configured to move the catheter proximally and distally. The drive system may be in communication with and controlled by the one or more control units. Also, the medical system may comprise a power generator coupled to and controlled by the one or more control units for providing electrical power to each of the plurality of electrodes.

Any of the systems or devices disclosed herein may have any of the following features. The distal portion of the catheter may be expandable and may include an interior portion and an exterior surface, and each of the plurality of electrodes may extend from the interior portion to the exterior surface.

A method of treating tissue may comprise positioning a distal portion of a catheter proximate to a treatment zone such that at least one electrode of a plurality of electrodes of the distal portion is adjacent to the treatment zone. The method may also comprise activating, via a control unit, the at least one electrode of the plurality of electrodes, to treat tissue of the treatment zone. The method may further comprise automatically moving the distal portion of the catheter relative to the treatment zone; and activating, via the control unit, at least one other electrode of the plurality of electrodes, to treat tissue of the treatment zone.

Any of the methods disclosed herein may include any of the following steps or features. The method may further comprise adjusting an amount of electrical energy supplied to at least one electrode of the plurality of electrodes based on a measured impedance of the at least one of the plurality of electrodes. The method may also comprise moving an internal component of the catheter relative to the distal portion to activate another electrode of the plurality of electrodes.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a schematic view of an exemplary medical ablation system, according to aspects of this disclosure.

FIGS. 2A-2D are side views of a portion of an exemplary medical device and various ablation shapes according to aspects of this disclosure.

FIG. 3 is a side view of a portion of an exemplary medical device positioned within a body lumen, according to aspects of this disclosure.

FIG. 4 is a side view of a portion of an exemplary medical device, according to aspects of this disclosure.

FIG. 5 is a front, cross-sectional view of a portion of an exemplary medical device, according to aspects of the present disclosure.

FIG. 6 is a side view of a portion of an exemplary medical device, according to aspects of this disclosure.

### DETAILED DESCRIPTION

The present disclosure is drawn to systems, devices, and methods for ablating, cutting, abrading, evaporating, or otherwise damaging or destroying tissue, among other aspects. Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.”

Embodiments of the present disclosure may be used to ablate tissue in an endo-luminal space, or facilitate the process thereof. In particular, some embodiments include an expandable or inflatable device including a plurality of electrodes. The device may be delivered to target tissue through an endoscope working channel or other structure for guiding the device, or may be delivered independently, without an endoscope, to the target tissue site. In some examples, the device may be fed distally from a proximal port, or back-fed, through an endoscope, gastroscope, colonoscope, flexible catheter, or other medical device working channel prior to inserting the device into the body of the patient. All or parts of the devices discussed herein could be metallic, composite, plastic, or include a shape memory metal (such as nitinol), a shape memory polymer, a polymer, or any combination of biocompatible materials.

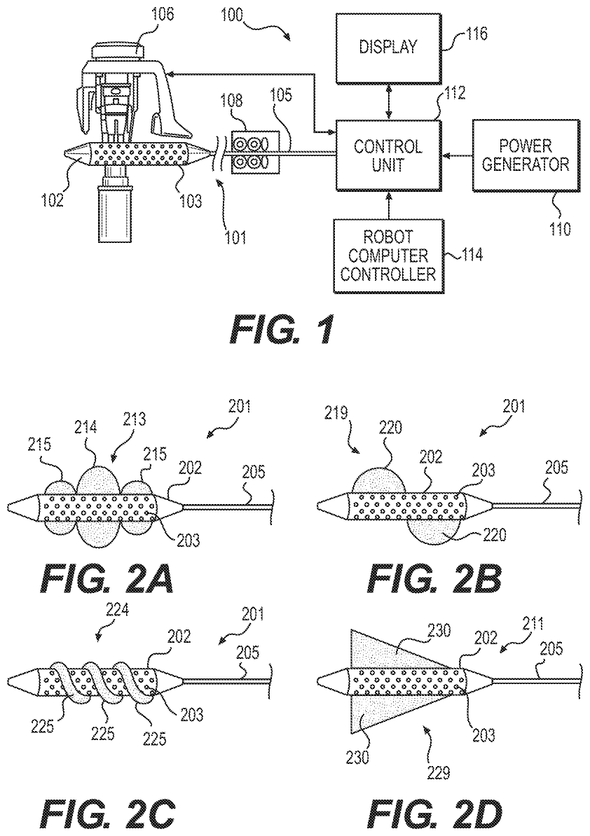

FIG. 1 shows an exemplary surgical system 100 in accordance with an embodiment of this disclosure. System 100 may include a catheter device 101, a scanner 106, a control unit 112, a power generator 110, a robot computer controller 114, a display 116, and a motor assembly 108. Catheter device 101 is configured to move through a body lumen of a patient, and ablate tissue using one or more electrodes 103 on an exterior surface of a distal portion 102 of catheter device 101. Catheter device 101 may be used during minimally invasive surgical procedures, such as laparoscopic or endoscopic procedures, or any other suitable medical procedure. Catheter device 101 may be used for radiofrequency ablation and may be configured to apply an electrical current produced by radio waves to tissue.

As shown in FIG. 1, catheter device 101 may include a distal portion 102, a proximal elongate 105, and one or more electrodes 103 positioned on a surface of distal portion 102. Distal portion 102 may be cylindrical and may have tapered, conical-shaped proximal and distal ends. The proximal end of distal portion 102 may be tapered radially-inward to a proximalmost end, and the proximalmost end may be coupled to proximal elongate. Distal portion 102 may be an expandable or inflatable body and may include a proximal lumen (not shown) connecting a lumen (not shown) of proximal elongate 105 to an interior cavity of distal portion 102. One or more electrodes 103 may be positioned on the exterior surface of distal portion 102. In some examples, a plurality of electrodes 103 may be positioned on the surface of distal portion 102 and may be each connected to control unit 112 through one or more wires (leads) positioned within proximal elongate 105. In some examples, each electrode 103 may be connected to control unit 112 through one or more wires or other electrical conductors printed on an interior surface of distal portion 102. In some examples, each electrode 103 may be individually controlled, and alternating electrodes 103 on distal portion 102 may be connected to apposing positive or negative poles. For example, distal portion 102 may be a bi-polar device with positive electrodes 103 adjacent to negative electrodes 103. In some examples, electrodes 103 may form a grid on the surface of distal portion 102. Electrodes 103 may form a pattern on the surface of distal portion 102 that may extend circumferentially about the longitudinal axis of distal portion 102. The pattern may include , for example, a plurality of longitudinal rows of electrodes 103, and a plurality of circumferential rings of spaced electrodes 103. In some examples, electrodes 103 may be circular, and/or distal portion 102 may include at least 5, 10, 15, 20, 24, 50, or 100 electrodes 103. In some examples, electrodes 103 may be evenly spaced in a grid pattern, such as in a grid pattern across part of or the entire radially-outer surface of distal portion 102 relative to the central longitudinal axis of distal portion 102. In some examples, electrodes 103 may be evenly spaced in a grid pattern across only the radially-outermost surface of distal portion 102 relative to the central longitudinal axis of distal portion 102. In some examples, each electrode 103 may protrude from the exterior surface of distal portion 102, and in other examples each electrode may be flush with the exterior surface of distal portion 102. The exterior surface of distal portion 102 may be flexible, compressible, and/or bendable, and may be configured to conform to irregular surfaces of a patient's anatomy. Each electrode 103 may be in communication with control unit 112 such that control unit 112 may monitor the impedance and other electrical characteristics of each electrode 103 and control power/current to each electrode 103.

Distal portion 102 may be inflatable or otherwise expandable, may include a compliant and/or a noncompliant material, and may be fluidly connected to a lumen (not shown) extending through proximal elongate 105. Air, saline, or another fluid may be input into the lumen to inflate distal portion 102. In other examples, distal portion 102 may be rigid. Proximal elongate 105 may be cylindrical and may be configured to translate, rotate, and otherwise move distal portion 102 through a body lumen. For example, proximal elongate 105 may be flexible and configured to bend through tortuous pathways of a body lumen, and may also be sufficiently rigid to translate distal portion 102 through a body lumen when proximal elongate 105 is translated distally. A proximal portion of proximal elongate 105 may be coupled to control unit 112.

Control unit 112 may be capable of interfacing with catheter device 101 to provide electrical current to the one or more electrodes 103 and monitor the impedance of each electrode 103. Control unit 112 may be coupled to, and in communication with, scanner 106, display 116, power generator 110, robot computer controller 114, motor 108, and/or catheter device 101. The control unit 112 may be powered by an external source such as an electrical outlet and/or power generator 110. Control unit 112 may include buttons, knobs, touchscreens, one or more graphical user interfaces, or other user interfaces to control one or more processors of control unit 112. In some examples, display 116 may provide a graphical user interface for control unit 112 and display 116 may consist of one or more monitors for displaying data received from control unit 112 or other devices of system 100. Control unit 112 may be configured to enable the user to set patterns of electrical stimulation to be applied to catheter device 101, such as by varying which electrodes 103 are electrified, adjusting the positioning of catheter device 101 via motor assembly 108, and/or applying pre-set electrical stimulation patterns to catheter device 101. For example, control unit 112 may be configured to activate and supply electrical power to groups of electrodes 103 depending on a user's or an algorithm's selection. In some examples, control unit 112 may be configured to adjust the electrical power supplied to each electrode 103 independently. Control unit 112 may be configured to receive and monitor information regarding the temperature, impedance, position, or other parameters of catheter device 101 or components of catheter device 101, such as one or more electrodes 103.

Motor assembly 108 may include one or more motors and may be configured to move catheter device 101 through a body lumen of a patient. Motor assembly 108 may include one or more rotational motors and one or more translational motors, and may be configured to receive a proximal portion of catheter device 101. Motor assembly 108 may be configured to move (including translate and/or rotate) catheter device 101 and may receive instructions from control unit 112. Robot computer controller 114 may be part of or separate of and connected to, control unit 112. In some examples, a user may interact with robot computer controller 114, such as via a mouse, knob, touchscreen, or other user interface, which relays instructions either directly to motor assembly 108 or through control unit 112 to motor assembly 108. In some examples, a user may insert a proximal portion of catheter device 101 through motor assembly 108 before coupling a proximal end of catheter device 101 to control unit 112. In some examples, motor assembly 108 may provide a means for robotically positioning catheter device 101 within a target area of a patient's body.

Scanner 106 may be a three dimensional computed tomography (CT) scanner, an ultrasound scanner, or any other type of scanner for scanning a patient's anatomy, taking images of a patients anatomy, and/or storing images of a patient's anatomy. Scanner 106 may be configured to image a treatment zone within a body of a patient, and output images to control unit 112 for display. In some examples, scanner 106 may be configured to detect catheter device 101 as catheter device 101 moves through a patient's body. Scanner 106 may be operatively coupled to control unit 112 so that control unit 112 receives real-time images during a procedure in which catheter device 101 is used. In some examples, scanner 106 may be configured to image an amount of ablation of a patient's tissue.

In some examples, a user may conduct a procedure using system 100 by first imaging a treatment zone within a patient's body using scanner 106. For example, a user may scan a patient's body using computed tomography (CT) scanning, and may generate three-dimensional images of a patient's anatomy including, for example, a body lumen. The user may then display, using control unit 112 and display 116, the three-dimensional images of the patient's anatomy via a graphical user interface (GUI). Once the treatment zone is identified in the images, the user may then select, using the GUI, an approximate volume of tissue for treatment (e.g. an approximate volume of tissue shown in the images to ablate). In some examples, control unit 112 may then select and implement the imaging thresholding, registration, and matrix transformations to segment areas of the selected target tissue. In some examples, imaging thresholding may include a method of identifying voxels between a certain color intensity (or threshold color intensity), and identifying clusters of voxels according to an algorithm for identifying shapes. Using imaging thresholding transformations, along with other image processing techniques known in the art, to identify a shape based on voxel and/or pixel color intensity may facilitate identification of the location of diseased tissue in a patient. In some examples, voxel and/or pixel color intensity may correlate to tissue density in an image produced by a CT scanner.

Image registration may include a method of associating a coordinate in three-dimensional space with each voxel in an image, for example by using an image from an initial scan. Subsequent scans creating subsequent images may then be compared to the initial scan, and the coordinates of each voxel in the images from subsequent scans may be compared to the coordinates of each voxel in the image from the initial scan, which may allow a user to identify where in three-dimensional space each voxel in a subsequent image is located. The method of image registration may also include applying a matrix transformation to obtain information on the translation and rotation of each voxel in space from an initial starting position shown in the initial scan image to a new position shown in an image from a subsequent scan. This method may be implemented by any image processing means known in the art. Image registration may be used to track the positioning of diseased tissue, among other aspects.

For example, control unit 112 may generate a graphical overlay of the desired treatment zone shown within one or more images of the patient's anatomy. Once the user has selected the treatment zone and the control unit 112 has calculated a volume of tissue to ablate, control unit 112 may calculate an ablation plan. An ablation plan may be a surgical plan for how to use system 100, and specifically catheter device 101, to ablate the treatment zone by specifying specific electrodes 103 of catheter device 101 to activate and specific amounts of electrical energy to be applied to each electrode once distal portion 102 is positioned proximate to or at the treatment zone. For example, the ablation plan may involve multiple overlapping ablations of varied shapes, depths and lengths. In some examples, the ablation plan aims to encompass all of the treatment zone while minimizing the amount of ablated healthy tissue. For example, the ablation plan may include instructions to activate a specific group of electrodes 103 in order to create a shaped ablation zone that targets unhealthy tissue of the treatment zone. In some examples, the ablation plan may include specific instructions for motor assembly 108 in order to position distal portion 102 at the treatment zone using motor assembly 108. The ablation plan may include instructions for the robot computer controller 114 to execute in order to position distal portion 102 of catheter device 101 at the treatment zone. In some examples, the user may confirm the ablation plan and may make adjustments to the ablation plan, as necessary, via the GUI.

When executing an ablation plan, the user may position distal portion 102 proximate to and/or at the selected treatment zone. For example, the user may align active portions, or portions at which electrodes 103 are positioned, of distal portion 102 with the treatment zone. The user may monitor the positioning of distal portion 102 using scanner 106, and may visualize via display 116 the positioning of distal portion 102 within the patient's body. In some examples, the control unit 112 may create and store a reference point, calculated using images generated by scanner 106, of the position of distal portion 102 at the treatment zone. The reference point, or reference position, may be an initial condition and/or an initial position of distal portion 102 created using an initial image from an initial scan of the treatment zone. In some examples, the reference point or reference position may be a starting position for a user to identify before treating the selected treatment zone. The reference point may be used by control unit 112 to calculate required movements of distal portion 102 relative to the treatment zone.

Once a reference point has been established and stored in control unit 112, control unit 112 may move catheter device 101 using motor assembly 108 to a starting point of treatment in accordance with the ablation plan outlined earlier. In some examples, control unit 112 may send instructions to motor assembly 108 to move catheter device 101 automatically, e.g. without human mechanical input from a proximal handle. Once the catheter device 101, and specifically distal portion 102, is positioned at the starting point, control unit 112 may activate power generator 110 and supply a specific group of electrodes 103 with energy at a predetermined power and voltage limit setting. By supplying the specifically selected electrodes 103 with the predetermined amount of energy, system 100 may create a shaped ablation similar to the planned shaped ablation established in the ablation plan. In some examples, control unit 112 may measure the real-time impedance feedback from each of the electrodes 103 and may actively adjust the energy supplied to each of the electrodes 103 based on the measured impedance feedback. In some examples, distal portion 102 of catheter device 101 may be moved after an initial shaped ablation is applied to the treatment zone, and then control unit 112 may supply a different, specifically selected group of electrodes 103 with a predetermined amount of energy. This process may be repeated until the entire treatment zone has been ablated. In some examples, control unit 112 may automatically calculate a new ablation plan based on measured impedance feedback from each of electrodes 103.

After ablation using catheter device 101, the user may then acquire CT or other medical images using scanner 106, and may compare the newly acquired images to the images used to create the ablation plan. The images showing the targeted tissue (such as diseased tissue) and the images showing the ablated tissue may then be registered to one another and compared to quantify the extent of ablation treatment, and confirm that all of the required tissue has been ablated. If portions of target tissue remain, the user may then create a new ablation plan to ablate the remaining tissue.

FIGS. 2A-2D illustrate various ablation patterns created by selecting specific electrodes 203 of a catheter device 201 to activate. Each ablation zone 214, 215, 220, 225, 230 may represent portions of tissue ablated, and each ablation zone 214, 215, 220, 225, 230 may be formed via regulation of electrical energy supplied to each electrode 203 and movement of distal portion 202. FIG. 2A shows catheter device 201 including distal potion 202, electrodes 203, proximal elongate 205, and an ablation pattern 213. Ablation pattern 213 includes a central region 214 and two lateral regions 215, with the central region 214 having the greatest ablation depth relative to the lateral regions 215. The radially-outermost edges of ablation pattern 213 are curved. The electrical energy supplied to each electrode 203 may be varied, and distal portion 202 may be moved, to form ablation pattern 213.

FIG. 2B shows catheter device 201 and ablation pattern 219 including an eccentric ablation zone 220 on opposite sides of catheter device 201. Ablation zone 220 may include two circular shapes positioned on opposite sides of distal portion 202 and include curved radially-outermost edges. Each portion of ablation zone 220 may be created by a different grouping of electrodes 203 of distal portion 202. Portions of ablation zone 220 may be semi-circular shaped.

FIG. 2C illustrates catheter device 201 and ablation pattern 224 including a helical shaped ablation zone 225. Ablation zone 225 may be formed by a plurality of electrodes 203 positioned around the surface of distal portion 202. Ablation zone 225 may wrap around distal portion 202, and, in some examples, may ablate portions of tissue extending circumferentially around a body lumen. Ablation patter 224 may be helical and/or cork-screw shaped.

FIG. 2D shows catheter device 201 and ablation pattern 229 including a gradient controlled ablation zone 230 that increases radially outward from the longitudinal axis of catheter device 211 as ablation zone 230 extends from a proximal end of distal portion 202 to a distal end of distal portion 202. A distal portion of ablation zone 230 may be larger relative to a proximal portion of ablation zone 230, and ablation zone 230 may form one or more triangular shapes. In some examples, ablation zone 230 may taper to a point at its one or more proximalmost ends. The energy applied to a distalmost electrode 203 may be greater than the energy applied to a proximalmost electrode 203 to form ablation zone 230. In other examples, an ablation pattern may include a proximal portion that is larger relative to a distal portion of the ablation pattern, and the ablation pattern may taper radially inward towards a central longitudinal axis of the catheter device as the ablation pattern extends distally.

FIGS. 2A-2D are exemplary, and a variety of different ablation patterns may be created using a plurality of electrodes 203 of catheter device 201 and regulating the energy output from each electrode 203. In addition, movement of catheter device 201, such as translation proximally, distally, or laterally, or rotation about its longitudinal axis, may allow catheter device 201 to create additional and varied ablation patterns. For example, part of an ablation plan may include rotating catheter device 201 about its longitudinal axis ninety degrees clockwise and ninety degrees counter clockwise, or other degrees of rotation in either direction.

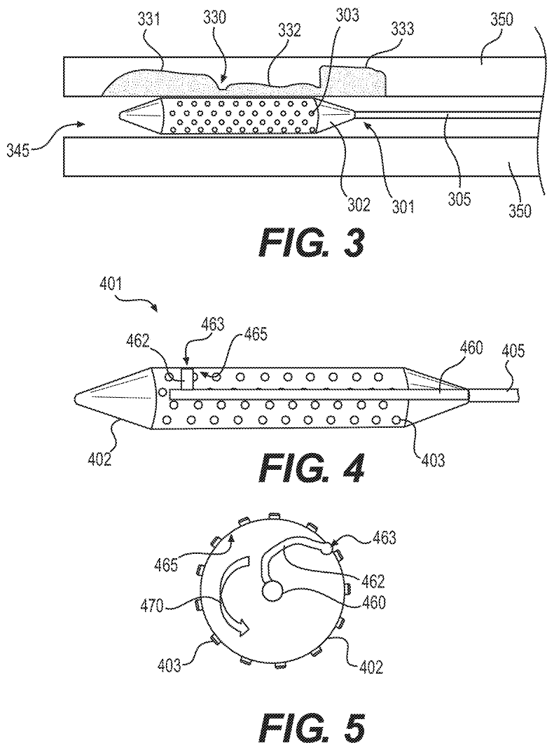

FIG. 3 shows a catheter device 301 including distal portion 302, electrodes 303, and proximal elongate 305, all of which are positioned within a body lumen 345 of a patient. Tissue 350 surrounding lumen 345 includes a target zone 330. A distal section 331 of treatment zone 330 requires a different depth and shape of ablation compared to intermediate section 332 and proximal section 333 of treatment zone 330. By regulating the amount of energy applied to each electrode 303 and moving catheter device 301 within lumen 345, a user may create an ablation pattern that aligns with treatment zone 330 and targets tissue of treatment zone 330 without damaging tissue adjacent to treatment zone 330. FIG. 3 depicts an example of an irregularly shaped treatment zone. The ability to selectively activate and adjust the energy omitted from a plurality of electrodes 303 of catheter device 301 provides the benefit of adjusting ablation patterns based on the user's and patient's needs.

FIG. 4 shows an alternative embodiment of a catheter device 401 including distal portion 402, a plurality of electrodes 403, and proximal elongate 405. Catheter device 401 may have any of the features described herein in relation to catheter devices 101, 201, 301. Catheter device 401 is substantially similar to catheter device 101, however each of electrodes 403 are not connected to individual corresponding wires to a control unit. Instead, each electrode 403 is commonly supplied power/current by an internal element 460 shared by electrodes 403. Internal element 460 may be cylindrical (e.g. a rod, wire, or the like), may be positioned within distal portion, and may extend through a lumen of proximal elongate 405. Internal element 460 may include a distal protrusion 462 extending radially outward from the longitudinal axis of internal element 460 at a distal end of element 460. A radially-outermost surface 463 of distal protrusion 462 may be configured to contact and slidably engage the interior surface 465 of distal portion 402. For example, rotation of internal element 460 about its longitudinal axis and/or translating internal element 460 proximally or distally may translate the radially-outermost surface 463 of distal protrusion 462 along interior surface 465 of distal portion 402 such that the radially-outermost surface 463 remains in contact with interior surface 465.

A proximal end of internal element 460 may be configured to couple to control unit 112 and may include an electrically conductive material to transfer electrical energy from control unit 112 to distal protrusion 462 of internal element 460. When the radially-outermost surface 463 of distal protrusion 462 contacts one or more electrodes 403, internal element 460 may transfer electrical energy supplied by control unit 112 to those one or more electrodes 403. For example, distal protrusion 462 may form an electrical connection with one or more electrodes 403 when distal protrusion comes into contact with an inner surface of the one or more electrodes 403. Internal element 460 may be moved proximally or distally and rotated about its longitudinal axis to locate specific electrodes 403 for electrical activation. In some examples, internal element 460 may continually translate proximally and/or distally and/or rotate at a specific frequency to create a user desired ablation pattern. In some examples (not shown), a catheter device may include an internal element (similar to internal element 460) with a plurality of protrusions (similar to protrusion 462) that may contact a plurality of electrodes simultaneously, and in some examples a catheter device may include a plurality of internal elements (similar to internal element 460) that may contact a plurality of electrodes simultaneously.

FIG. 5 illustrates a front view of a cross-section C of catheter device 401. Arrow 470 illustrates the rotation of proximal protrusion 462 about the longitudinal axis of internal element 460. Distal protrusion 462 may be curved, as shown in FIG. 5, and may form a C-shape. In some examples, distal protrusion 462 may be rigid and in other examples distal protrusion 462 may be flexible. Each electrode 403 may include a radially-inward facing surface that remains exposed to the interior space of distal portion 402 during operation of catheter device 401 to allow a radially-outermost surface 463 of distal protrusion 462 to directly contact each electrode 403.

Catheter device 401 may operate in substantially the same manner as catheter device 101 described hereinabove. In some examples, a proximal portion of internal element 460 may be coupled to a motor assembly separate from a motor assembly used to control the position of distal portion 402 and proximal elongate 405. By activating each electrode 403 using internal element 460, catheter device 401 may not require additional wiring from each electrode 403 and may facilitate manufacturing and miniaturization of catheter device 401.

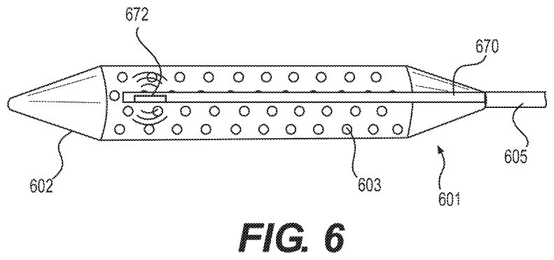

FIG. 6 shows another alternative embodiment of a catheter device 601 including distal portion 602, a plurality of electrodes 603, and proximal elongate 605. Catheter device 601 may have any of the features described herein in relation to catheter devices 101, 201, 301, 401. Catheter device 601 may include an ultrasound probe 672 coupled to an internal member 670 positioned within an interior portion of catheter device 601. Ultrasound probe 672 may be positioned within an interior portion of distal portion 602 and may emit an ultrasound signal. Ultrasound probe 672 may be electrically coupled with and in communication with control unit 112, such as through a wire extending through an interior portion of internal member 670. In operation, a signal emitted from ultrasound probe 672 may allow a user to monitor the position of distal portion 602 within a body of a patient by ultrasound imaging. For example, scanner 106 may include an ultrasound scanner and may be used to monitor the position of distal portion 602 within a patient's body during a procedure. By using ultrasound probe 672 when positioning distal portion 602 of catheter device 601 at a treatment zone within a patient's body, the user may confirm the location of distal portion 602 using ultrasound imaging. In some examples, ultrasound probe 672 may enable a user to create a three-dimensional view of the ablation of a patient's tissue using ultrasound imaging techniques.

By providing a catheter device that a user may selectively ablate tissue and specifically regulate power applied to a plurality of electrodes positioned at a treatment zone, a user may reduce injury of healthy tissue and avoid unnecessary harm to a patient's body caused by the excessive ablation of tissue during a radiofrequency ablation procedure.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

1-15. (canceled)

16. A medical system, comprising:
a catheter for ablating tissue including:
a flexible longitudinal body including a distal end; and
a distal portion extending distally from the distal end of longitudinal body, the distal portion including a plurality of electrodes; and
one or more control units coupled to the catheter and configured to (1) control a supply electrical energy to each of the plurality of electrodes and (2) automatically control a position of the distal portion of the catheter.

17. The system of claim 16, further comprising:
a drive system configured to move the catheter proximally and distally, wherein the drive system is in communication with and controlled by the one or more control units.

18. The system of claim 16, further comprising:
a power generator coupled to and controlled by the one or more control units for providing electrical energy to each of the plurality of electrodes; and
a scanner configured to create images of a patient's anatomy.

19. The system of claim 16, wherein the one or more control units are configured to monitor an impedance of each of the plurality of electrodes and adjust the electrical energy supplied to each of the plurality of electrodes based on the monitored impedance.

20. The system of claim 16, further comprising a graphical user interface configured to allow a user to select an area of tissue targeted for ablation by the plurality of electrodes.

21. The system of claim 16, wherein the one or more control units are configured to adjust an amount of electrical energy supplied to at least one of the plurality of electrodes based on at least one image created by the scanner.

22. The system of claim 16, wherein the one or more control units include a plurality of stored ablation patterns, wherein each stored ablation pattern includes output energy levels for each of the plurality of electrodes.

23. The system of claim 16, wherein the catheter includes an internal element extending from a proximal portion of the catheter to the distal portion, and wherein:
the internal element includes a distal protrusion with a radially-outermost surface in contact with a radially-inner surface of the distal portion,
the internal element is positioned within, and moveable relative to, the distal portion and the longitudinal body, and
the internal element is configured to transfer electrical energy to each of the plurality of electrodes independently of others of the plurality of electrodes.

24. The system of claim 16, wherein the catheter includes an ultrasound probe positioned within the distal portion;
wherein the scanner is configured to detect the position of the ultrasound probe.

25. The system of claim 16, wherein the distal portion of the catheter is expandable and includes an interior portion and an exterior surface, wherein each of the plurality of electrodes extends from the interior portion to the exterior surface.

26. The system of claim 16, wherein the distal portion of the catheter is cylindrical and includes a conical distal portion and a conical proximal portion; and
wherein the plurality of electrodes form a grid pattern around the radially-outermost portion of the distal portion.

27. The system of claim 23, wherein the distal protrusion is configured to activate each of the plurality of electrodes independently when in contact with each electrode, and wherein the distal protrusion is configured to translate longitudinally and rotate relative to the distal portion.

28. The device of claim 27, wherein each of the plurality of electrodes is not connected to a proximal lead; and
wherein the distal protrusion is curved.

29. The system of claim 17, wherein the drive system includes a plurality of motors to translate the catheter longitudinally and to rotate the catheter about a longitudinal axis of the catheter.

30. The system of claim 16, wherein the one or more control units is configured to independently supply electrical energy to each of the plurality of electrodes.

31. A medical system comprising:
a catheter for ablating tissue including:
a flexible longitudinal body including a distal end; and
a distal portion extending distally from the distal end of longitudinal body, the distal portion including a plurality of electrodes;
one or more control units coupled to the catheter and configured to (1) supply electrical energy to each of the plurality of electrodes independently and (2) automatically control a position of the distal portion of the catheter;
a drive system configured to move the catheter proximally and distally, wherein the drive system is in communication with and controlled by the one or more control units; and
a power generator coupled to and controlled by the one or more control units for providing electrical power to each of the plurality of electrodes.

32. The system of claim 31, wherein the distal portion of the catheter is expandable and includes an interior portion and an exterior surface, wherein each of the plurality of electrodes extends from the interior portion to the exterior surface.

33. A method of treating tissue, the method comprising:
positioning a distal portion of a catheter proximate to a treatment zone such that at least one electrode of a plurality of electrodes of the distal portion is adjacent to the treatment zone;
activating, via a control unit, the at least one electrode of the plurality of electrodes, to treat tissue of the treatment zone;
automatically move the distal portion of the catheter relative to the treatment zone; and
activate, via the control unit, at least one other electrode of the plurality of electrodes, to treat tissue of the treatment zone.

34. The method of claim 33, further comprising adjusting an amount of electrical energy supplied to at least one electrode of the plurality of electrodes based on a measured impedance of the at least one of the plurality of electrodes.

35. The method of claim 34, further comprising moving an internal component of the catheter relative to the distal portion to activate another electrode of the plurality of electrodes.
